(19) United States
(12) Patent Application Publication
(10) Pub. No.: US 2020/0276387 A1
(43) Pub. Date: Sep. 3, 2020

(54) A SYSTEM FOR ASSISTING IN ADMINISTRATION OF A DRUG

(71) Applicant: Novo Nordisk A/S, Bagsvaerd (DK)

(72) Inventors: Prakarn Nisarat, Seattle, WA (US); Kirk Cornell, Seattle, WA (US)

(21) Appl. No.: 16/647,272

(22) PCT Filed: Aug. 30, 2018
(86) PCT No.: PCT/EP2018/073299

§ 371 Date: Mar. 13, 2020

Related U.S. Application Data
(60) Provisional application No. 62/561,810, filed on Sep. 22, 2017.

(30) Foreign Application Priority Data
Oct. 4, 2017 (EP) 17194658.5

(51) Int. Cl. A61M 5/172; A61M 5/315

(52) U.S. Cl. CPC A61M 5/1723; A61M 2005/3126; A61M 5/31568; A61M 5/31533

(57) ABSTRACT

A system and kit for assisting a subject in administering an injectable drug by use of an injection device having communication means to communicate data at least about sizes of doses expelled and a timestamp for when each dose was expelled. The system comprises a communication unit (210) adapted to communicate with the injection pen (100) to receive said dose data and comprising a display to present information and instructions to the subject. The system has a processor unit (220) comprising one or more processors and a memory storing a dose size calculation program adapted calculate a recommended dose size of drug to be injected. Responsive to a request to calculate a recommended dose size, the memory stores instructions that, when executed by the one or more processors, performs the initial step of prompting the user to perform a priming of the injection device before a calculation of a recommended dose size can be carried out.

### FIELD OF THE INVENTION

The present disclosure relates to a system and kit for assisting a subject in managing injections of a drug for treatment of a disease such as diabetes.

### BACKGROUND OF THE INVENTION

Type 2 diabetes mellitus is characterized by progressive disruption of normal physiologic insulin secretion. In healthy individuals, basal insulin secretion by pancreatic 3 cells occurs continuously to maintain steady glucose levels for extended periods between meals. Also in healthy individuals, there is prandial secretion of insulin in response to a meal, followed by prolonged insulin secretion that returns to basal levels after 2-3 hours. The insulin secretion is impaired in Type 2 diabetes and for patients with Type 1 diabetes there is very low to no insulin secretion at all, why Type 1 and 2 patients require insulin injections to control their glucose level.

A common approach to diabetes treatment is to inject a single short acting insulin medicament (bolus) dosage in accordance with a standing insulin regimen for the patient in response to or in anticipation of a meal event using one insulin pen. In such approaches, the patient injects the short acting insulin medicament dosage shortly before or after one or more meals each day to lower glucose levels resulting from such meals. Examples of such short acting insulin medicaments include, but are not limited, to Lispro (HUMALOG, insulin lispro [rDNA origin] injection, Eli Lilly and Company) and Aspart (NOVOLOG, insulin aspart [rDNA origin] injection, NOVO NORDISK Inc). Further, the patient injects a long acting insulin medicament (basal) dosage in accordance with the standing insulin regimen, independent of meal events, to maintain glycaemic control independent of meal events. Examples of such long acting insulin medicaments include, but are not limited to Insulin Degludec or Insulin Detemir. An example of a mixture of insulin medicaments with a collective long duration of action is Insulin Degludec/Insulin Aspart combination. An example of a mixture of insulin medicaments with other medicaments that collectively have a long duration of action is Insulin Degludec/Liraglutide combination.

The most common type of injection delivery system for insulin treatment is insulin injection pens, which can be used to self-administer insulin medicament treatment regimens. Some insulin injection pens today are able to register data about the size of doses being expelled, time of injection and type of insulin and communicate this data wirelessly from the pen to another device, such as a smart phone. This in combination with having glucose measuring devices, such as the glucose sensor FREESTYLE LIBRE Flash Glucose Monitor by ABBOTT (“LIBRE”), that makes autonomous glucose level measurements of a patient and wirelessly upload glucose data to a device, means that patients today have access to devices/systems that collect these data and are able to provide the patients with insulin dose size guidance. All this for the purpose of helping patients in their self-treatment of the disease and keep them in better glycaemic control while not needing to consult their health care practitioner often.

Such devices/systems today includes algorithms that are able to calculate and recommend a dose of insulin to be injected based e.g. on historic glucose data and insulin data. In order to ensure correct dose calculation, such system requires up-to-date dose data history which is transferred from the insulin pen (or an insulin pump) before each dose calculation. As these device/systems give specific medicine treatment guidance, it is obviously crucial that the calculation is as correct as possible and based on as accurate historic data as possible. Especially for diabetes, hazards relate to overdosing—if the algorithm suggests a higher dose of insulin than is safe it could lead to hypoglycaemia and potentially dangerous situations.

Today patients on pen-based treatment are requested to prime their insulin pen(s) before dialling the dose of insulin to be injected, this in order to remove any air bubbles in the insulin cartridge or needle of the pen and to make sure that whatever dose the patient dials on the pen is also what is injected into the body. Normally the patient is requested to expel 2-3 units of insulin as a priming dose. The pen will register this prime dose as a regular dose event, as the pen is not able to distinguish between different types of doses. When the dose data then is uploaded to another device or server for further analysis, dose calculation etc. this prime dose will be registered as an injected dose. However, for the dose calculations mentioned above, it is highly important that the calculation is based on true injected insulin doses and not priming doses, and if said prime dose is included in the set of historic injected insulin doses, first of all the tracking of the patients therapy will be wrong but also future dose recommendation calculations will be wrong, which can have serious negative impact for the patient.

Due to the above, in order to give as correct dose recommendations as possible, the system needs to distinguish between primed and actual doses to be able to disregard the prime doses in the calculation. Traditionally, this has been done with user input, i.e. the user has to mark each dose event as either “primed” or “injected” dose right after each injection. However, this creates friction for user experience where there are chances of users either forgetting to mark the dose type after each dose expelling or mark the doses wrongly.

Different solutions for how to distinguish between prime and injected doses have been proposed, for example as described in WO 2016/007935. However, with the solutions disclosed in this document mistakes can still happen, i.e. the system distinguishes wrongly or the patient categorises the doses wrongly or even forget to categorise it.

Other background references are WO2013/156510, WO2012/127046, WO2014/020008 and an article “A novel pen-based Bluetooth-enabled insulin delivery system with dose tracking and advice” by Timothy S. Bailey and Jenine Y. Stone (2017), Exp. Op. Drug Del. 14, 697-703.

Having regard to the above, it's an object of the present invention to provide an improved and safer way of filtering out non-injected dose of drug, such as prime doses, in the set of data being transferred from a connected injection device.

It's a further object of the present invention to provide a system and kit for assisting patients in administering their injections and wherein the risk of the patient injecting a different dose size than what was recommended and/or dialled on the device is minimised.

It should be understood that, although the terms first, second, etc. may be used herein to describe various elements, these elements should not be limited by these terms. These terms are only used to distinguish one element from another. Furthermore, the terms “subject,” “user,” and “patient” are used interchangeably herein.

### SUMMARY OF THE INVENTION

Accordingly, a system for assisting a subject in administering an injectable drug by use of an injection device having communication means to communicate data at least about sizes of the doses expelled and a timestamp for each expelled dose, said system comprises a communicatior unit adapted to communicate with the injection device to receive said data and comprising a display to present information and instructions to the subject, and a processor unit comprising one or more processors and a memory storing a dose size calculation program adapted calculate a recommended dose size of drug to be injected. Said memory further stores instructions that, when executed by the one or more processors, performs the method step of, responsive to a request to calculate a recommended dose size to be injected; prompting the subject, via said display, to perform the step of priming said injection device by setting and expelling a predefine prime dose size in order to allow execution of said program to calculate a recommended dose size.

By providing a system requiring the patient to perform the prime dose (sometimes also called an “Air Shot”) as the first initial step in order to allow execution of the program to calculate a dose size, there is no chance that the patient forget to prime the device and thus the potential risk of the patient getting a wrong insulin dose, e.g. due to air bubbles in the device, is completely mitigated. Said predefined prime dose size may be indicated in said display at the same time as prompting the user to perform the step of priming.

The memory further stores instructions that, when executed by the one or more processors, performs the method steps of, subsequent to the step of prompting the subject to prime the device; obtaining a first set of dose data from the device about sizes of the expelled doses, then processing said first set of dose data to identify said prime dose in the data set, then filtering out the prime dose data from the first set of dose data to provide a second set of dose data.

The memory further stores instructions that, when executed by the one or more processors, performs the method steps of calculating by said program, based upon said second set of dose data, a recommended dose size to be injected, and presenting said recommended dose size to the subject via said display.

It's a very simple and safe filtering of dose data leaving no chance of distinguishing wrongly between the different doses expelled from the device. The identification of the prime dose may be carried out in the processor unit.

The step of identifying the prime dose preferably comprises selecting as the prime dose the latest registered expelled dose size out of the number of expelled dose sizes in the first set of dose data.

The fact that the patient MUST do the priming of the device before the system will start calculating a recommended dose size combined with this simple filtering out of the last dose means that there is no need for relying on the patient's manual input or alternatively a complex algorithm for distinguishing between different expelled doses, which have a high chance of giving a false categorisation of the different doses in the first set of dose data.

As appears from above, by the system according to the invention a calculation of a recommended dose size will always be based on true historic injected doses NOT including any prime doses, as the dose calculation only will start after the device has been primed and the primed dose has been filtered out from the set of data upon which the calculation is done. Thereby, the patient is always ensured that the recommended dose size is calculated on as correct historic data as possible and that the recommended dose size that he/she subsequently dials on the device and injects is also what is really injected, as the device already has been primed, i.e. no air bubbles etc. in the device.

Furthermore, the invention gives added benefits such as instead of the patient being prompted multiple times to mark a prime dose from actual injected doses, as proposed in some prior art systems, the system handles this automatically when it receives the dose data from device. The more automatically the system operates for the patient the better, as one should bear in mind that being a diabetic means dealing with many devices, taking decisions, logging data etc. every day, which can be stressful and burdensome. Furthermore, patients are more likely to perform the priming of the device (which is highly important as stated above) when being prompted to do so by the system than they normally are. This is due to the instant benefit and value proposition of a dose guidance patients receive in exchange for the priming step. Without this benefit the patients would view the priming step both as waste of time but also as a waste of drug.

Instead of identifying the latest expelled dose as the prime dose, the system may alternatively identify the prime dose as the smallest registered expelled dose size out of the number of expelled dose sizes in the first set of dose data.

Alternatively, the step of identifying the prime dose may comprise comparing the expelled dose sizes of the first set of dose data with said predefined dose size and selecting the expelled dose in the first set of data that has a dose size identical to the predefined dose size as the prime dose.

As part of requesting a dose size recommendation, the patient may be prompted via the display to indicate what type of drug he/she requests a dose recommendation for, this for safety reasons. To ensure that the drug type in the device is the right type, the first set of dose data may further include drug type data indicating what kind of drug there is contained in the device (fx long or short acting insulin). In that case the memory may store instructions that, when executed by the one or more processors, further performs the method steps of, prior to the step of calculating a recommended dose; processing said first set of dose data to identify the type of drug in the primed device, then comparing the type of drug in the primed device with the type of drug for which a recommended dose is requested, and only if the two types of drug are identified as identical, then executing said program to calculate a recommended dose size.

By this the patient is always ensured that whatever drug that he/she requested a dose size recommendation for is also what is in the device at hand. If there is any inconsistence the system will, via the display, inform the patient that a dose recommendation will not be calculated, as the drug type on the device is wrong.

Other typical issues that come with insulin therapy may be mitigated by the system according to the invention for the benefit of the patients, both as to user convenience and safety. For example if the device is broken, the battery is about to run out (e.g. meaning no dose data history can be transferred from the device), the outlet/needle is blocked or other dysfunctionalities in device, then the first set of data obtained from the device may include data informing about such errors in the device etc.

It should be noted that any drug suitable for injection by use of an injection device is within the scope of the invention. The drug may be insulin, insulin containing drug, a GLP-1 containing drug, growth hormone or any other injectable drug. If its insulin then the predefined prime dose size is usually 3 units or less, such as 2 units or 1 unit of insulin.

The communication unit may be a smartphone (e.g. Android or IOS) but could also be a tablet or computer. The processor unit with one or more processors and memory storing the dose size calculation program may either be located on the same smartphone, tablet or computer, but is preferably located on a cloud-based server separate from the communication unit.

In an embodiment the communication unit is adapted to receive, from a glucose measuring device measuring the subject's glucose level, glucose data about historic glucose levels and timestamps for when each glucose level was measured, and wherein said calculation of a recommended dose size to be injected further is based upon said glucose data.

The communication unit may communicate wirelessly with the injection device, processor unit and glucose measuring device via NFC, Bluetooth, BLE (Bluetooth Low Energy), Wi-Fi, ZigBee, GSM or Narrow Band.

A second aspect of the invention provides a kit for assisting a subject in administering a drug and comprising a system as described in connection with the first aspect and an injection device for injecting doses of drug into the body of the subject. The device comprises a drug reservoir, an outlet for the drug, a dose setting mechanism, a dose expelling mechanism to expel set doses of drug through the outlet, a dose sensing unit for sensing the size of doses being set and/or expelled, and communication means to communicate dose data from the device about at least sizes of the expelled doses.

Another aspect of the invention provides a system for providing a recommended dose size of drug to be injected into the body of a subject by use of an injection device having communication means to communicate data about sizes of the expelled doses from the device. The system comprises a communication unit adapted to communicate with the injection device to receive said data and comprising a display to present information and instructions to the subject, a processor unit comprising one or more processors and a memory storing a dose size calculation program adapted calculate a recommended dose size of drug to be injected. The memory further stores instructions that, when executed by the one or more processors, performs the method step of, responsive to a request to calculate a recommended dose size to be injected; prompting the subject, via said display, to perform the step of priming said injection device by setting and expelling a predefined prime dose size indicated in the display in order to allow execution of said program to calculate a recommended dose size.

A further aspect of the invention provides a method for providing a recommended dose size of drug to be injected into the body of a subject. The method comprises;
using a system comprising;
   a communication unit adapted to communicate with the injection device to receive data about expelled dose sizes and comprising a display to present information an instructions to the subject,
   a processor unit comprising one or more processors and a memory, said one or more processors including a dose size calculation program adapted calculate a recommended dose size of drug to be injected,
said memory stores instructions that, when executed by the one or more processors, performs the step of, responsive to a request to calculate a recommended dose size to be injected;
prompting the subject, via said display, to perform the step of priming said injection device by setting and expelling a predefined prime dose size indicated in the display in order to allow execution of said program to calculate a recommended dose size,
obtaining a first set of dose data from the device,
processing said first set of dose data to identify said prime dose in the data set,
filtering out the prime dose data from the first set of dose data to provide a second set (dose data,
calculating, based upon said second set of dose data, a recommended dose size to be injected, and
presenting said recommended dose size to the patient via said display.

In a further aspect is provided a computer program comprising instructions that, when executed by a computer having one or more processors and a memory, performs the method step of, responsive to a request to calculate a recommended dose size of drug to be injected into the body of subject by an injection device;
prompting the subject to perform the step of priming said injection device by setting and expelling a predefined prime dose size in order to initiate a calculation of a recommended dose size,
obtaining a first set of dose data from the device,
processing said first set of dose data to identify said prime dose in the data set,
filtering out the prime dose data from the first set of dose data to provide a second set of dose data,
calculating, based upon said second set of dose data, a recommended dose size to be injected, and
presenting said recommended dose size to the patient via said display.

In an even further aspect is provided a computer-readable data carrier having stored there on the computer program as described above.

According to any of the aspects above, the step of calculating a recommended dose may be based on a prescribed insulin medicament dosage regimen that specifies an algorithm for computing and recommending a dose size based upon a data set comprising one or more blood glucose (BG) measurements taken over a time course, and for each respective measurement in the plurality of measurements, a corresponding timestamp representing when in the time course the respective measurement was made, and one or more injected drug dose sizes taken over a time course, and for each respective injected dose size in the plurality of injected doses, a corresponding timestamp representing when in the time course the respective dose was injected. Said data set may also include historic data of insulin sensitivity factor (ISF) estimations, Insulin on board (IoB) estimations, carbohydrates on board (COB) estimations, historic geographical positions, cardiovascular activity, food intake, calendar events, heart rate, skin temperature, skin impedance and/or respiration, and for each of the respective measurements a corresponding timestamp representing when in the time course the respective measurement was made.

### BRIEF DESCRIPTION OF THE DRAWINGS

FIG. 1 illustrates a diagram of an exemplary embodiment of the system according to the invention,

FIG. 2 shows a flow diagram of an exemplary method of requesting, calculating and registering a dose size of insulin using the system and kit according to the invention, and

FIG. 3 shows examples of screen layouts of a user interface of the system according to the invention.

### DETAILED DESCRIPTION

In the following detailed description, more specific details are set forth in order to provide a more thorough understanding of the present disclosure. However, it will be apparent to one of ordinary skill in the art that the present disclosure may be practiced without these specific details.

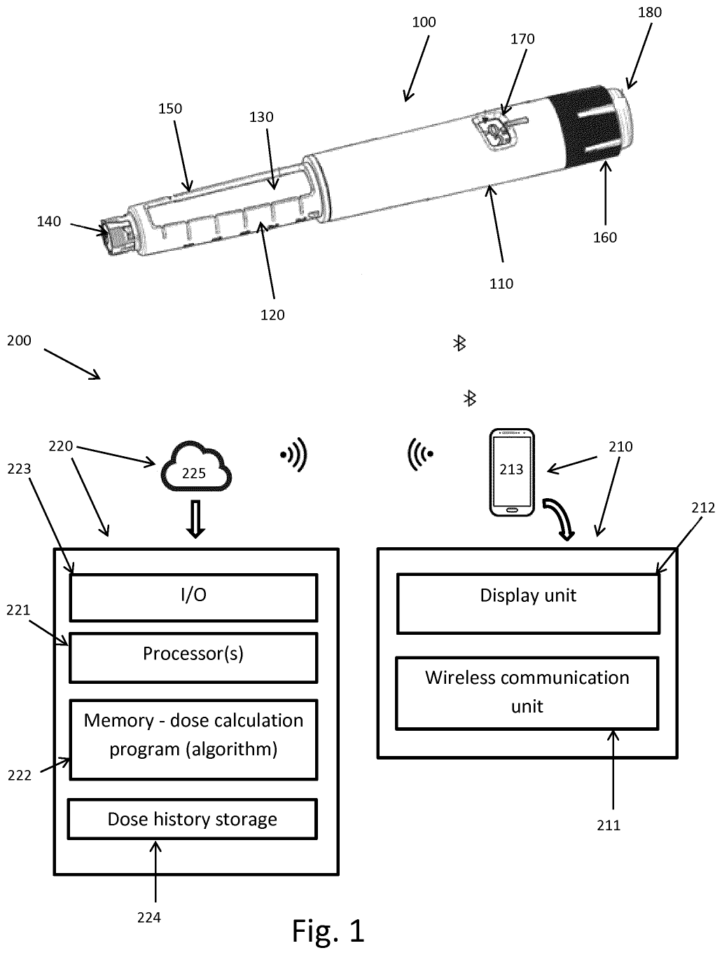

In FIG. 1 an example of an injection device in the form of a pen 100 used for taking discrete doses of drug is shown. Any injection pen (durable or disposable) able to register/log and communicate data from the pen is applicable to the invention described herein.

More specifically, the pen 100 comprises a main part having a proximal body or drive assembly portion with a housing 110 in which a drug expelling mechanism is arranged or integrated to expel set doses of drug through an outlet, and a distal drug reservoir holder portion 120 holding a drug-filled transparent drug reservoir in the form of a cartridge 130 having a displaceable piston (not shown) and distal needle-penetrable septum 140. The cartridge holder has openings 150 allowing a portion of the cartridge to be inspected. The cartridge may for example contain a insulin, GLP-1 or growth hormone formulation. A proximal-most rotatable dose ring member 160 serves as dose setting mechanism to manually set a desired dose of drug shown in display window 170 and which can then be expelled when a release button 180 is actuated. The pen further comprises a dose sensing unit (not shown) for sensing and registering the size of doses being expelled, and communication means to communicate dose data from the pen, in this example via Bluetooth®.

FIG. 1 further shows a system 200 according to the invention adapted to communicate with the injection pen 100 to receive data about sizes of doses expelled from the pen, timestamps associated with each of the expelled doses, type of drug in the pen etc. The pen 100 may further communicate information about status of the pen, such as battery status, any dysfunction in the pen, blocked outlet/needle etc.

The system 200 comprises a communication unit 210, in this example a mobile phone 213, adapted to communicate via a wireless communication unit 211 with the injection pen 100. The communication unit 210 further comprises a display unit 212 to present information and instructions to the user.

The system further comprises a processor unit 220 including one or more processors 221 and a memory 222 storing the dose calculation program adapted to calculate a recommended dose size of drug to be injected upon request from the user. The processor unit 220 further comprises an input/output module 223 for transferring data to and from the communication unit 210. The dose calculation program preferably includes dose calculation algorithm for the specific type of drug, patient and/or other parameters.

The processor unit 220 further comprises a dose history storage unit 224 for storing the historic dose data received from the injection pen 100 via the communication unit 210.

As depicted in the figure, the processor unit 220 is located on a server in the cloud, 225, remote from the communication unit 210 and communicates with the communication unit 211 via Wi-Fi. Alternatively, the communication unit 210 and processor unit 220 may be included in one single unit, such as a smartphone, tablet or computer.

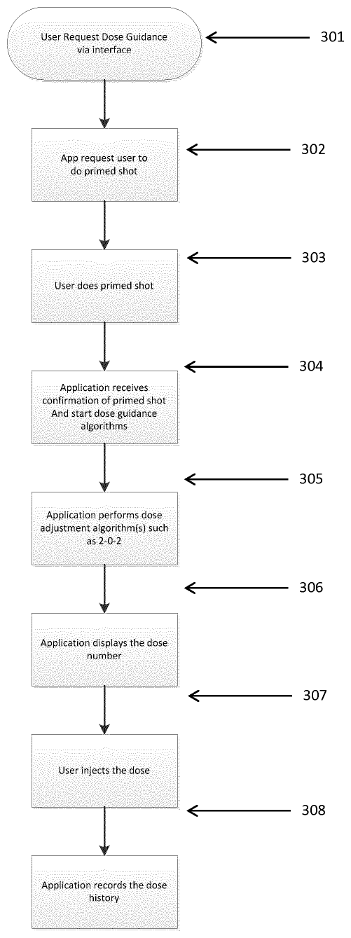

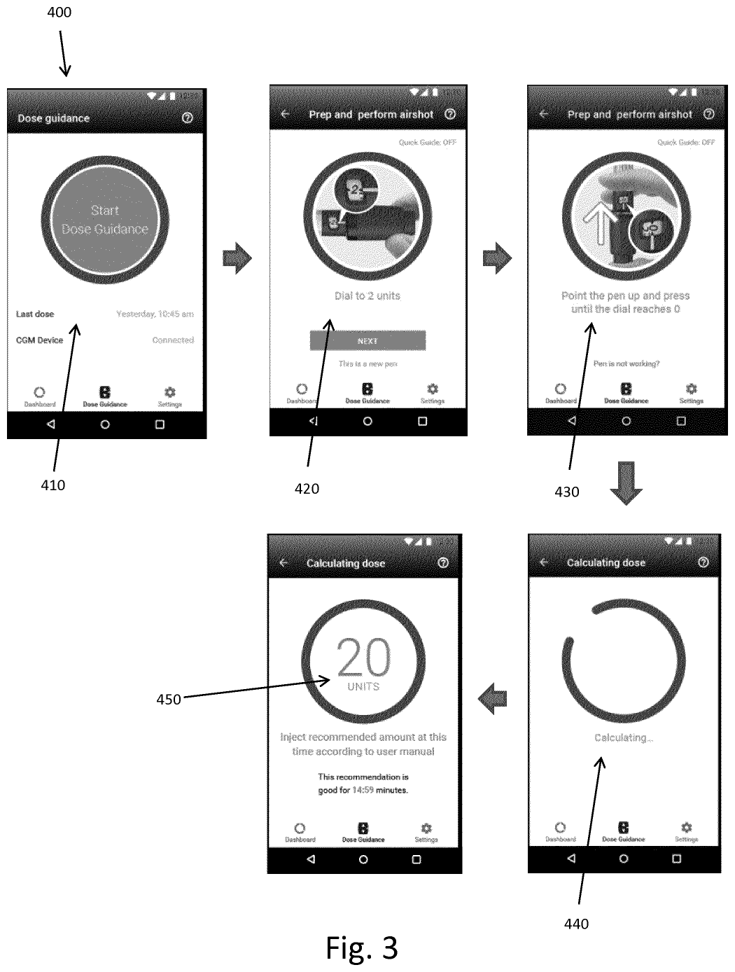

Turning to FIG. 2 a flow diagram of an exemplary method of requesting, calculating and registering a dose size of insulin using the system according to the invention is shown. The flow starts by the user requests, 301, a dose calculation of a recommended dose size via a display interface on the communication unit, such as the interface on the mobile application as the shown in FIG. 3. In order to trigger a dose size calculation, the communication unit now requests, 302, via the display the user to prime the injection pen by expelling a predefined no. of units of insulin from the pen (a primed shot). The user then dials up said predefined no. of units on the pen, activates the expelling mechanism and expels the prime dose, 303.

The communication unit now obtains a first set of dose data from the pen and transfer that set of data further on to the processor unit located in the cloud. The processor unit confirm that a prime dose has been expelled and filters out the prime dose from the first set of dose data as described above and a calculation of a recommended dose can now take place, 304. A dose calculation program is executed calculating a dose based upon a second set of dose data not including the prime dose 305.

When the recommended dose size has been calculated and sent from the processor unit to the communication unit, the dose size is displayed for the patient on the display of the communication unit, 306. Next the patient will set the recommended dose size on the injection pen and inject it, 307. After the injection has taken place, the communication unit receives updated dose data from the pen, transfers the data to the of processor unit, where the updated dose data history, 308, is stored in a dose history storage unit.

In FIG. 3 are shown examples of user interfaces and associated required user steps for receiving a dose size recommendation in a system according to the invention.

The user starts opening the dose calculation application 400 on the smartphone, which can be any kind of smartphone (Android/IOS). A first screen 410 pops up where the user can hit the “Start Dose Guidance” button to start a dose calculation.

The next screen 420 pops up requesting the user to prepare the injection pen for a prime dose (in the figure called “airshot”) by dialling 2 units on the dose setting mechanism on the pen.

Now the screen 430 pops up instructing the user to activate the dose expelling mechanism on the pen by pressing the button while pointing the pen upwards and wait until “0” is shown in the unit window.

As soon as the user releases the button, a first set of dose data is sent from the pen via the mobile phone to the processor unit, in this case the mobile phone, and the 2 units prime dose will immediately be filtered out and not recorded as injected dose. The dose calculation algorithm starts calculating a dose size recommendation 440 based on the recorded second set of dose data not including the 2 units prime dose.

Finally, the screen 450 pops up displaying the calculated recommended dose size to the user, and the user injects the recommended dose (or other amount based on user's discretion). As soon as the user releases the button on the pen after having injected the dose, the dose data is sent via the mobile phone to the processor unit and the injected dose is registered in the dose data history records.

The first set of data may further include drug type information that allows the application to verify that the pen contains the correct type of drug that the user has requested a dose size recommendation for. If not, then the system will not proceed to the dose size calculation step but inform the user of the mismatch and maybe ask the user to take the pen containing the correct type of drug.

The data from the pen may further include information codes about any errors in the pen (pen is broken, injection needle/outlet blocked, low battery etc.). In the event of an error, the system will relay error message(s) to the user via the display based on error codes received from the injection pen and no dose calculation will be made.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A system for assisting a subject in administering an injectable drug by use of an injection device having communication structure to communicate data at least about sizes of doses expelled and a timestamp for when each dose was expelled from the device,
said system comprising;
a communication unit adapted to communicate with the injection device to receive said data and comprising a display to present information and instructions to the subject,
a processor unit comprising one or more processors and a memory storing a dose size calculation program adapted calculate a recommended dose size of drug to be injected,
said memory further storing instructions that, when executed by the one or more processors, performs the method step of, responsive to a request to calculate a recommended dose size to be injected;
a) prompting the subject, via said display, to perform the step of priming said injection device by setting and expelling a predefined prime dose size in order to allow execution of said program to calculate a recommended dose size.

2. A system according to claim 1, wherein the memory further stores instructions that, when executed by the one or more processors, performs the method steps of, subsequent to the step of prompting the subject to prime the device,
b) obtaining a first set of dose data from the device containing said data about at least sizes of the doses expelled and a timestamp for when each expelled dose was expelled,
c) processing said first set of dose data to identify said prime dose in the data set, and
d) filtering out the prime dose data from the first set of dose data to provide a second set of dose data.

3. A system according to claim 2, wherein the memory further stores instructions that, when executed by the one or more processors, performs the method steps of, subsequent to the step of filtering out the prime dose data,
e) calculating by said program, based upon said second set of dose data, a recommended dose size to be injected, and
f) presenting said recommended dose size to the subject via said display.

4. A system according to claim 2, wherein the step c) of identifying the prime dose comprises selecting as the prime dose the latest registered expelled dose size out of the number of expelled dose sizes in the first set of dose data.

5. A system according to claim 2, wherein the first set of dose data further includes drug type data, said memory storing instructions that, when executed by the one or more processors, further performs the method steps of, prior to the step of calculating a recommended dose;
i. processing said first set of dose data to identify the type of drug in the primed device,
ii. comparing the type of drug in the primed device with the type of drug for which a recommended dose is requested,
and only if the two types of drug are identified as identical, then
iii. executing said program to calculate a recommended dose size.

6. A system according to claim 1, wherein the drug is insulin, insulin containing drug, a GLP-1 containing drug or growth hormone.

7. A system according to claim 1, wherein the communication unit is a smartphone, tablet or computer.

8. A system according to claim 7, wherein the one or more processors and memory is provided on said smartphone, tablet or computer.

9. A system according to claim 1, wherein the one or more processors and a memory are provided on a cloud-based server remote from the communication unit.

10. A system according to claim 1, wherein the communication unit is adapted to receive, from a glucose measuring device measuring the subject's glucose level, glucose data about historic glucose levels and timestamps for when each glucose level was measured, and wherein said calculation of a recommended dose size to be injected further is based upon said glucose data.

11. A kit for assisting a subject in administering a drug and comprising;
an injection device for injecting doses of drug into the body of the subject, said device comprising;
a drug reservoir,
an outlet for the drug,
a dose setting mechanism,
a dose expelling mechanism to expel set doses of drug through the outlet,
a dose sensing unit for sensing the size of doses being set and/or expelled, and communication structure to communicate dose data from the device about at least sizes of the expelled doses and timestamps for each expelled dose from the device,
and a system according to claim 1.
